(19) United States
(12) Patent Application Publication
(10) Pub. No.: US 2026/0226399 A1
(43) Pub. Date: Aug. 6, 2026

(54) Methods for Producing and Cultivating High Productivity Algae Strains

(71) Applicants: Global Algae Technology, LLC, San Diego, CA (US); Pacific Northwest National Laboratory, Richland, WA (US); Colorado School of Mines, Golden, CO (US)

(72) Inventors: David A. Hazlebeck, San Diego, CA (US); Agnieszka Pinowska, San Diego, CA (US); Jesse Traller, San Diego, CA (US); Alexander Beliaev, Richland, WA (US); Eric Hill, Richland, WA (US); Matthew Posewitz, Golden, CO (US); Tyson Burch, Golden, CO (US)

(21) Appl. No.: 19/152,867

(22) PCT Filed: Feb. 5, 2024
(86) PCT No.: PCT/US2024/014519

§ 371 Date: Jul. 31, 2025

Related U.S. Application Data
(60) Provisional application No. 63/443,298, filed on Feb. 3, 2023.

(51) Int. Cl. C12N 1/12

(52) U.S. Cl. CPC C12N 1/12

(57) ABSTRACT

The present disclosure describes a method for producing high oxygen tolerant algae strains. The methods include selectively growing algae cell cultures under high oxygen pressure conditions. Algae strains produced by these methods, as well as methods for cultivating them, are also disclosed.

### CROSS-REFERENCE TO RELATED APPLICATIONS

This application claims the benefit of U.S. Provisional Application No. 63/443,298 filed on Feb. 3, 2023, the content of which is incorporated by reference in its entirety.

### STATEMENT OF FEDERALLY SPONSORED RESEARCH OR DEVELOPMENT

This invention was made with government support under award #DE-EE0008245 awarded by the Department of Energy (“DOE”) and under sub-recipient award #401301-5802 and under award #DE-AC05-76RL01830 awarded by the DOE. The government has certain rights in the invention.

### BACKGROUND OF THE DISCLOSURE

Algae cultivation has become widely recognized as a promising source of food, feed, biofuel, chemicals, polymers and nutraceuticals. Photosynthetic algae production offers the potential for an order of magnitude higher agricultural productivity than other plants. Algae is cultivated in photobioreactors. There are open photobioreactor such as open raceway ponds, closed photobioreactors such as glass or plastic tubes, and attached growth photobioreactors such as algae fixed surfaces in or out of the water.

Unfortunately, the economics of large-scale cultivation remains challenging. One way to improve the economics is through obtaining higher productivity in the cultivation systems, so numerous approaches have been taken to improve the productivity of in various cultivation systems. Reduced algal growth rates from oxygen inhibition is a well-known problem in cultivation of algae for commercial products (Marquez et al.). As a result, many efforts have been made to reduce the dissolved oxygen content of photobioreactors (Weissman et al.).

Some algae strains are preferred because of higher lipid content, better lipid profiles, or higher value compounds. Examples of some strains of commercial interest are Nitzschia for lipid content and profile; Haematococcus for astaxanthin content; Nannochloropsis and a variety of diatoms for EPA content; and Spirulina for phycocyanin and protein content. Typically, eukaryotic algae can achieve higher lipid content than cyanobacteria (procaryotic algae), so these strains are preferred for lipid-based products such as biofuel, vegetable oils, and polymers. These species all exhibit oxygen tolerance limitations on growth rate.

Photosynthetic or mixotrophic algae growth requires a supply of carbon dioxide. If the pH is lower than 9, then carbon dioxide will off-gas from cultivation media into air at a high rate. Therefore, ideally cultivation systems are operated at a pH of 10 or higher to facilitate capture of carbon dioxide from the air rather than loss of carbon dioxide to the air. At neutral pH, bioreactors require many more carbon dioxide introduction points, and loss of carbon dioxide is very high. As a result, the cost of supplying carbon dioxide can be 30 to 60 percent of the cultivation costs. This issue is exacerbated by the need to remove dissolved oxygen. Greater air-liquid mixing to remove dissolved oxygen increases the loss of carbon dioxide if the pH is less than 9 to 10.

While the problem of lower productivity has been widely recognized over 70 years, and many attempts have been made to develop higher productivity strains, there have been very few attempts to evolve photosynthetic or mixotrophic algal strains with higher oxygen tolerance, and no attempts for commercially applicable strains have been made. The only successful algae evolution for oxygen-tolerance was in a non-commercial strain and was done to try to understand the mechanism of oxygen tolerance (Wagner et al.). In this case, the algae only grew with a high carbon dioxide concentration gas bubbled into the media, which lowered the pH to about 8. The strain did not grow at all with air bubbled into the media, which would have increased the pH to over 9.5. Recently, a few strains of algae have been found that are more tolerant of high oxygen concentrations in neutral pH environments (Gao et al.); however, algal strains have not been evolved or discovered that achieve higher oxygen tolerance at pH of 9 or greater for commercial applications.

### SUMMARY OF THE DISCLOSURE

The disclosure provides novel algae strains and methods of producing and cultivating them.

In an aspect of the present disclosure, provided herein is a method of photosynthetically or mixotrophically cultivating a high oxygen tolerant algae strain, the method comprising cultivating the strain in an aqueous medium comprising a pH of greater than 9.

In some embodiments, the aqueous medium pH during cultivation is greater than 10. In some embodiments, the strain has less than an about 40% reduction of growth rate at about 24 mg/L of dissolved oxygen concentration relative to the growth rate at about 8 mg/L dissolved oxygen concentration. In some embodiments, the strain has less than an about 20% reduction of growth rate at about 16 mg/L of dissolved oxygen concentration relative to the growth rate at about 8 mg/L dissolved oxygen concentration. In some embodiments, the high oxygen tolerant algae strain has less than an about 40% reduction of growth rate at about 24 mg/L of dissolved oxygen concentration relative to the growth rate at about 8 mg/L dissolved oxygen concentration.

In some embodiments, the aqueous medium comprises at least 0.1 molar concentration of carbonate and bicarbonate ions combined. In some embodiments, the carbonate comprises at least one of sodium carbonate and potassium carbonate, and wherein the bicarbonate comprises at least one of sodium bicarbonate and potassium bicarbonate.

In some embodiments, the high oxygen tolerant algae strain is cultivated in a photobioreactor.

In another aspect, provided herein is a Nitzchia inconspicua strain GAI-337.

In another aspect, provided herein is a method of producing a high oxygen tolerant algae strain is provided, the method comprising: a) growing a cell culture of a parent algae strain in a medium comprising a pH of at least 9.0; b) gradually increasing oxygen concentration in the medium while diluting slow-growing cells out of the medium until cell growth is inhibited; c) decreasing the oxygen concentration in the medium; d) allowing the cell growth to recover; and e) repeating steps a) through d) at least one time; wherein after step e), the cell culture comprises the oxygen tolerant algae strain.

In some embodiments, steps a) through d) are repeated one time.

In some embodiments, the light intensity during the cell culture growth is greater than the saturating intensity of the parent algae strain. In some embodiments, the cell culture concentration is low enough to prevent self-shading. In some embodiments, the pH of the medium is between 9 and 11. In some embodiments, the method is done in a photobioreactor. In some embodiments, the method is done in a turbidostat.

In some embodiments, the high oxygen tolerant algae strain has less than an about 40% reduction of growth rate at about 24 mg/L of dissolved oxygen concentration relative to the growth rate at about 8 mg/L dissolved oxygen concentration. In some embodiments, the high oxygen tolerant algae strain has less than an about 20% reduction of growth rate at about 16 mg/L of dissolved oxygen concentration relative to the growth rate at about 8 mg/L dissolved oxygen concentration.

In some embodiments, the parent algae strain is a wild-type strain. In some embodiments, the parent algae strain is a Nitzschia strain. In some embodiments, parent algae strain is Nitzschia inconspicua.

In another aspect, provided herein is a high oxygen tolerant algae strain produced by the disclosed methods.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1 presents the relative growth rate versus dissolved oxygen concentration for a wild-type strain of Nitzschia inconspicua, GAI-229.

FIG. 2 presents the relative growth rate versus dissolved oxygen concentration for a high oxygen tolerant strain of Nitzschia inconspicua, GAI-337.

FIG. 3 presents the expected (circles) and actual (diamonds) improvement in growth rate of the oxygen tolerant strain over the wild-type at high pH in a photobioreactor.

FIG. 4 presents the average growth rate in a photobioreactor under conditions typical of commercial-scale systems. Comparison of average areal productivity of ash-free biomass (AFDW) in g m-2 d-1 for GAI-229 (wild-type) and GAI-337 (mutant) under high oxygen stress diel-cycle conditions described in the Methods is shown. Results are for biological triplicates of GAI-229 over four consecutive days (n=12) and for biological duplicates of GAI-337 over four consecutive days (n=8). Error bars represent ±1 standard deviation. Statistical comparison of means was done by t-test (two-tailed, two-sample homoscedastic).

FIG. 5. presents the productivity of the improved strain with carbon dioxide in the air bubbled into the bioreactor and without carbon dioxide in the air bubbled into the bioreactor. Mean areal productivity in g AFDW m⁻² d⁻¹ is shown for biological triplicate cultures of GAI-337 supplemented with 0.5% CO₂ (n=24) and a single culture grown with air (n=8) over 8 consecutive days. Cultures were grown under high-productivity pond-mimicking conditions with an OD₇₅₀ setpoint of 1.25 after dilution. Error bars represent ±1 standard deviation of the mean productivities.

FIG. 6. illustrates a turbidostat capable of implementing the method of the present disclosure for developing high oxygen tolerant algae strains.

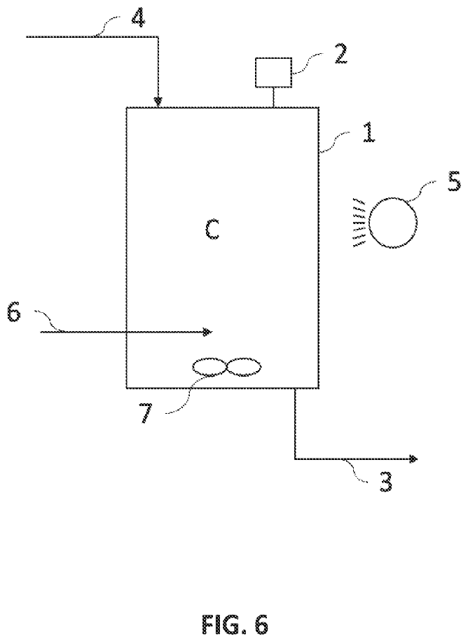

FIG. 7 illustrates the cycling oxygen concentration and growth rate during evolution of Nitzschia inconspicua to obtain a higher oxygen tolerant strain. Growth rate per hour (left axis, determined by dilution rate per hour) and dissolved O₂ (right axis, in % of air saturation) over time during the generation of the high O₂ tolerant strain GAI-337 from the wild-type strain GAI-229. The grey line represents the dilution rate hr⁻¹ taken every 10 minutes, the solid dark line represents the average growth rate (dilution rate) smoothed out by taking the five hour average of 1 hour moving averages of dilution rates, and the dashed line represents the measured dissolved O₂ concentration.

FIG. 8 shows a comparison of growth rate per hour at three dissolved O₂ concentrations under constant light (960 μmol photons m⁻² s⁻¹), temperature (24° C.), and pH (9.2), in a turbidostat photobioreactor for the wild-type strain GAI-229 (light grey) and the high-oxygen strain GAI-337 after one round of high O₂ selection (dark grey), and GAI-337 after two rounds of high O₂ selection (black).

FIGS. 9A-9B show dissolved oxygen (blue) and temperature (red) over the course of a diel cycle on a hot summer day in outdoor ponds (A) and in our pond mimicking bottles during maximal areal productivity (B).

FIG. 10 shows areal productivity in g AFDW m⁻² d⁻¹ for GAI-337 as dependent upon OD₇₅₀ after dilution. Cultures were grown under pond-mimicking conditions, sampled and diluted just after sunset. Each OD₇₅₀ setpoint was sampled for three consecutive days in biological triplicate. Each point represents one sample, and the line is a polynomial line of best fit with equation and R² indicated.

FIG. 11 shows a comparison of areal productivities in g AFDW m-2 d-1 for GAI-337 under different air flow rates and maximum diel-cycle temperatures (24° C. at night, temperature curve mimicking an outdoor pond with the maximum temperature indicated during the day). Cultures were grown under pond-mimicking diel light in biological triplicate for three consecutive days (n=9) for each condition shown. Error bars represent ±1 standard deviation.

FIGS. 12A-12D show a time series of OD750 (A), pH just prior to dilution (B), AFDW in g L-1 (C), and areal productivity in g AFDW m-2 d-1 (D) for GAI-337 under pond-mimicking conditions. One culture was grown with air only (grey lines), and biological triplicate cultures were grown with air supplemented with 0.5% CO2 (black lines), both conditions were bubbled at a rate of 100 mL min-1 per bottle with the gas indicated. Pond-mimicking light and temperature (23-33° C.) were provided to the cultures. The OD750 initial setpoint after dilution was set to 0.8 for days 0 to 10, then 1.25 thereafter. The triplicate bottles supplemented with 0.5% CO2 were switched to lipid induction medium for the final three days of the experiment. Error bars represent ±1 standard deviation of biological triplicates.

### DETAILED DESCRIPTION

The present invention has been described in terms of one or more preferred embodiments, and it should be appreciated that many equivalents, alternatives, variations, and modifications, aside from those expressly stated, are possible and within the scope of the invention.

Provided herein is a method of rapidly evolving an algae strain having improved intrinsic growth rate at higher dissolved oxygen concentration relative to its wild-type or parent strain through selective pressure. In embodiments, the method comprises growing a cell culture of the parent algae strain, and gradually increasing dissolved oxygen concentration while diluting slow-growing cells out of the culture until the concentration of dissolved oxygen is high enough to inhibit the cell growth. The dissolved oxygen concentration is then lowered until the algae cell growth recovers. This process is then repeated at least one time, to produce a high oxygen tolerant algae strain. In some embodiments, the process is repeated only once. The step of growing the cell culture of the parent algae strain may be performed under conditions of high light. The light intensity during the step of growing the cell culture of the parent algae strain may be greater than the saturating intensity of the parent algae strain. The cell culture of the parent algae strain may be grown in a medium comprising a pH of between about 9.0 and about 11.

In some embodiments, the step of gradually increasing the oxygen concentration in the medium is performed until cell growth rate is inhibited by at least 80%. The step of lowering the oxygen concentration may be performed until the cell growth recovers to its initial rate.

In embodiments, the step of gradually increasing the dissolved oxygen is performed by increasing the oxygen by about 100% or about 8 mg/L every 6 to 12 hours.

The parent algae strain used in the disclosed methods may be a wild-type algae strain. In some embodiments, the strain is a Nitzschia strain. In exemplary embodiments, the strain is Nitzschia inconspicua. Other algae strains may be used, such as Spirulina strains and Chlorella strains, such as Chlorella sorokiniana.

In some embodiments, the disclosed methods produce a high oxygen tolerant algae strain that has a modest reduction growth rate at elevated oxygen concentrations. A modest reduction is defined as less than 20% reduction in growth rate at an oxygen concentration of 200% of saturation and less than 40% reduction in growth rate at an oxygen concentration of 300% saturation relative to the growth rate at 100% saturation. The dissolved oxygen concentration at saturation is the equilibrium oxygen concentration under atmospheric conditions or approximately 8 mg/L, so the concentration at 200% of saturation is approximately 16 mg/L and at 300% of saturation is approximately 24 mg/L.

The methods disclosed herein may further comprise photosynthetically or mixotrophically cultivating the high oxygen tolerant the strain in an open or closed photobioreactor with at least one of a bicarbonate and a carbonate in the culture media such that the pH is greater than 9, so that a much higher productivity is obtained relative to similar species or strains with low oxygen tolerance.

Also provided herein are high oxygen tolerant algae strains produced by the disclosed methods.

An advantage of the present disclosure is increased productivity in mixotrophic and photosynthetic cultivation of algae in open and closed photobioreactors.

Another advantage is increased productivity in mixotrophic and photosynthetic cultivation of algae in open and closed photobioreactors with high carbon dioxide utilization efficiency by operating at a pH 9 or higher.

Another advantage is increased productivity in mixotrophic and photosynthetic cultivation of algae in open and closed photobioreactors with direct air capture of carbon dioxide by operating at pH of 10 or higher.

Another advantage is increased productivity in mixotrophic and photosynthetic cultivation of algae in open and closed photobioreactors by utilizing the combination of a high oxygen tolerant strain and cultivation at greater than pH 9 with carbonates and bicarbonate in the media.

Another advantage is rapid development of new high oxygen tolerant algae strains.

As used herein, the term “wild-type” refers to the most common of an algae strain found in nature. As used herein, the terms “parent strain” or “parental strain” refer to an algae strain cultivated to produce a new algae strain. In some embodiments, the parent strain is the wild-type strain of the algae.

As used herein, the term “mixotrophic cultivation” or “mixotrophy” refers to a mode of cultivation, where heterotrophic and autotrophic modes work simultaneously, leading to utilization of inorganic and organic carbon in the presence of light.

As used herein, the term “photosynthetic cultivation” refers to a mode of cultivation in which an autotrophic mode uses inorganic and organic carbon in the presence of light.

### Miscellaneous

The present invention has been described in terms of one or more preferred embodiments, and it should be appreciated that many equivalents, alternatives, variations, and modifications, aside from those expressly stated, are possible and within the scope of the invention.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. The term “consisting essentially of” and “consisting of” should be interpreted in line with the MPEP and relevant Federal Circuit interpretation. The transitional phrase “consisting essentially of” limits the scope of a claim to the specified materials or steps “and those that do not materially affect the basic and novel characteristic(s)” of the claimed invention. “Consisting of” is a closed term that excludes any element, step or ingredient not specified in the claim.

As used in this specification and the claims, the singular forms “a,” “an,” and “the” include plural forms unless the context clearly dictates otherwise. For example, the term “a substituent” should be interpreted to mean “one or more substituents,” unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

The invention will be more fully understood upon consideration of the following non-limiting examples.

The present invention has been described in terms of one or more preferred embodiments, and it should be appreciated that many equivalents, alternatives, variations, and modifications, aside from those expressly stated, are possible and within the scope of the invention.

The following Examples are offered for illustrative purposes only and are not intended to limit the scope of the present invention in any way. Indeed, various modifications of the invention in addition to those shown and described herein will become apparent to those skilled in the art from the foregoing description and the following examples and fall within the scope of the appended claims.

### EXAMPLES

The following Examples are illustrative and should not be interpreted to limit the scope of the claimed subject matter.

### Example 1

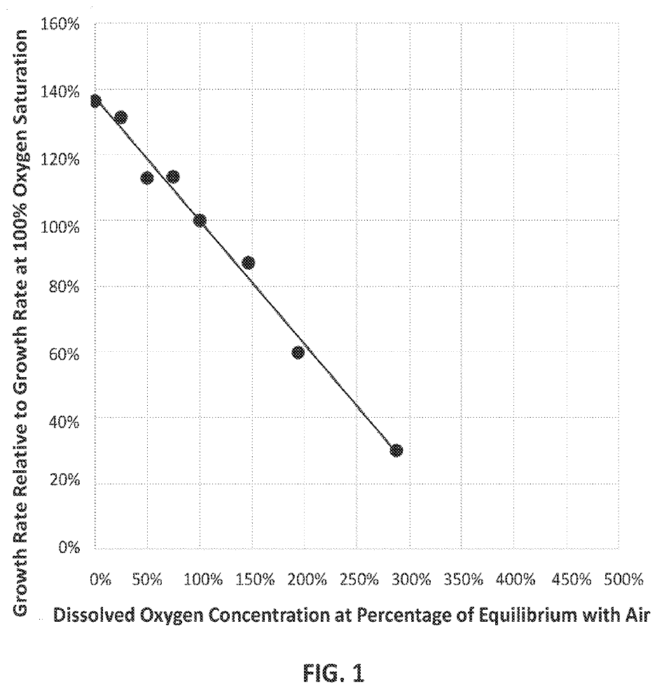

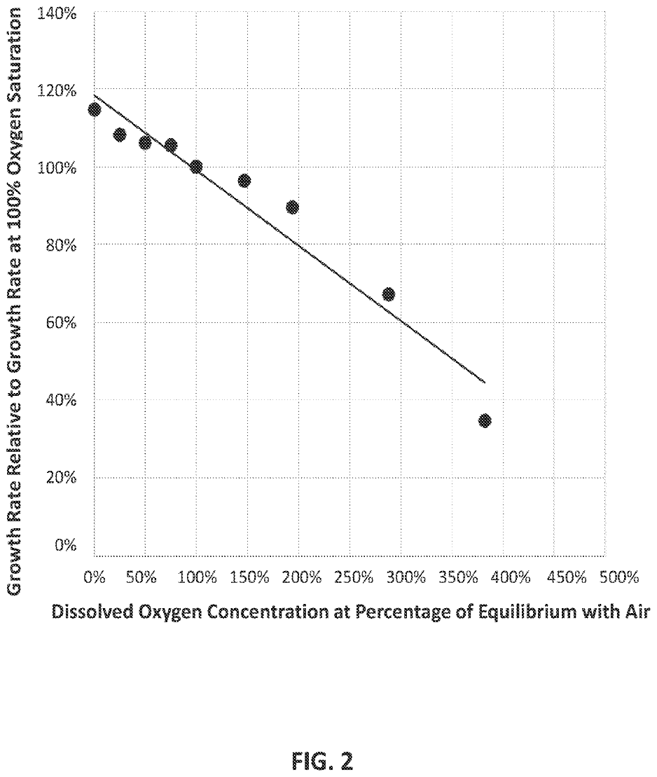

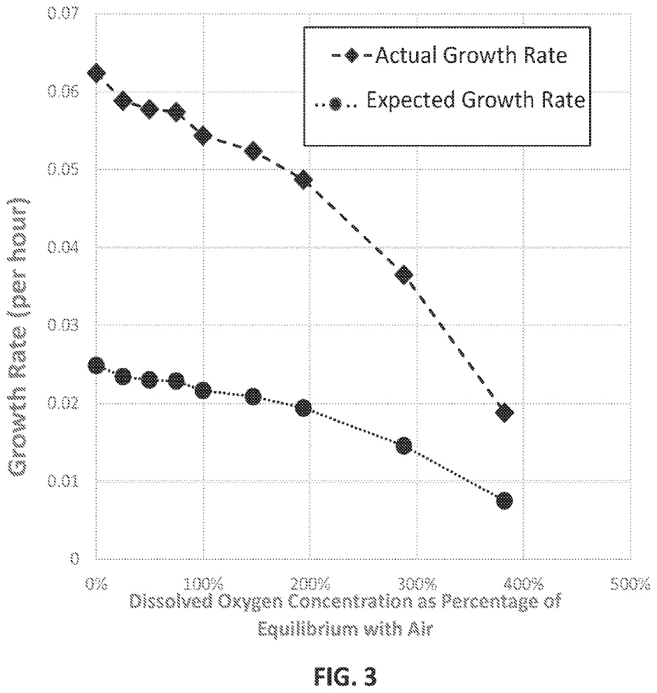

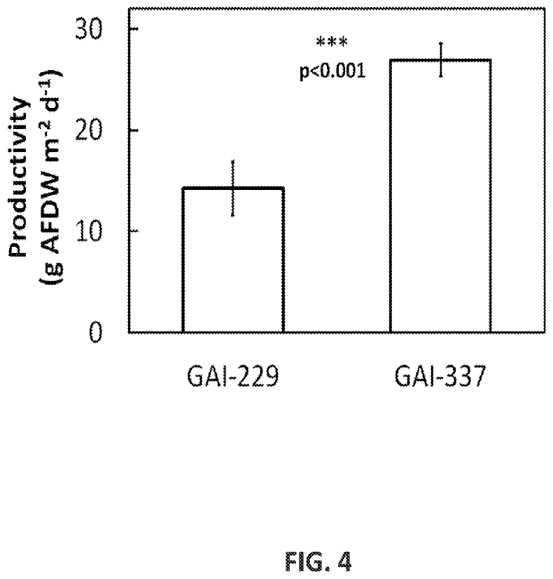

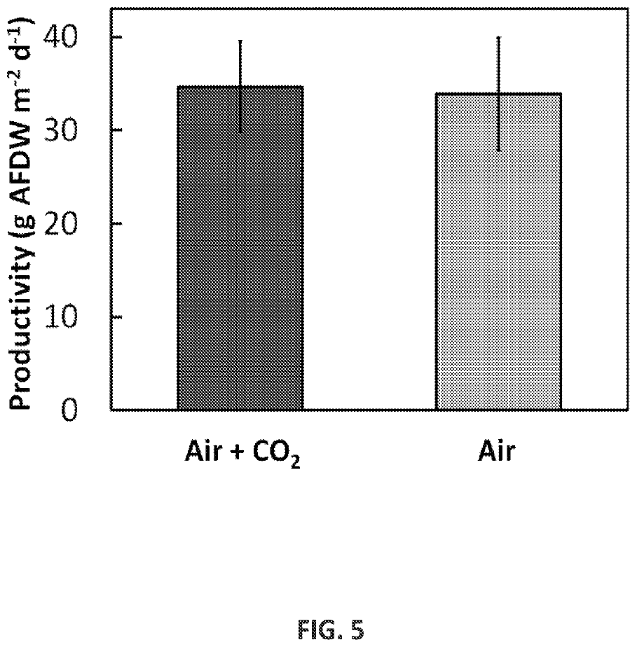

A wild-type Nitzschia inconspicua strain, GAI-229, has a 40% reduction in growth rate at 200% oxygen saturation and a 70% reduction in growth rate at 300% oxygen saturation as illustrated in FIG. 1. An improved Nitzschia inconspicua strain, GAI-337, was developed that only has a 10% reduction in growth rate at 200% oxygen saturation and a 32% reduction in growth rate at 300% oxygen saturation as illustrated in FIG. 2. When the wild-type and improved strains were grown at a pH of 9.3 in a 0.3 molar sodium solution of bicarbonate and carbonate, the improvement in growth rate was much higher than could be accounted for by the improved oxygen tolerance. There was a large, unexpected benefit of growing the higher oxygen tolerant algae strain at high pH relative to a less oxygen tolerant strain. FIG. 3 presents the projected growth rate from the for the improved strain based on the higher oxygen tolerance and the actual growth rate at pH 9.3. The wild-type and oxygen tolerant strains were tested in a photobioreactor under typical large-scale cultivation conditions. The productivity was 90% greater in the GAI-337 strain, under typical large-scale cultivation as illustrated in FIG. 4. As illustrated in FIG. 5, the high oxygen tolerant strain, GAI-337, productivity in a photobioreactor with 0.3M sodium as a mixture carbonate and bicarbonate bubbled with 0.5% carbon dioxide in air where the equilibrium pH is approximately 9.3 was the same to the growth rate with no added carbon dioxide in the air where the equilibrium pH is approximately 10.

FIG. 5 illustrates a photobioreactor turbidostat that can be used for adaptive laboratory evolution of algae strains for high oxygen tolerance. The algae is grown in the photobioreactor, 1, at concentration, C, monitored by the optical density meter, 2. Culture is withdrawn through a harvest conduit, 3, and fresh media added through media conduit, 4, so maintain a constant concentration of algae in the turbidostat. Light source, 5, illuminates the turbidostat for photosynthetic of mixotrophic growth. A gas mixture is bubbled into the turbidostat to control the dissolved oxygen concentration in culture media through an air input conduit, 6. Heating and cooling can be added to control the temperature of the turbidostat, and the light can be adjusted to control the light intensity. The algae culture is mixed from air bubbling or from an internal mixer, 7, inside the turbidostat so that culture is well-mixed. Algae typically reach photosaturation at about 200 microEinsteins per meter squared per second (μmE/m2/s) of light intensity. If the culture is concentrated, then it will self-shade and higher incident light than 200 μmE/m2/s will lead to higher growth rate because each cell is experiencing a lower average light intensity. For dilute cultures, a light intensity higher than about 200 μmE/m2/s is considered high light because there is little self-shading.

Continuous operation of the turbidostat will select for the fastest growing cells. A non-limiting method of generating algae strains with high oxygen tolerance is to perform continuous growth algae in a turbidostat under high light conditions in a dilute culture; increase the dissolved oxygen concentration until the algae stop growing due to oxygen inhibition; and then reduce the dissolved oxygen concentration so the so that the algae continue growing. This process can be performed a single time or multiple times in series.

Similar conditions can be used in other bioreactors such as in flasks, carboys, plates, or other photobioreactor configurations. The essential steps are to maintain the algae under a high growth selection and to increase the oxygen concentration until the algae stop growing.

### Example 2

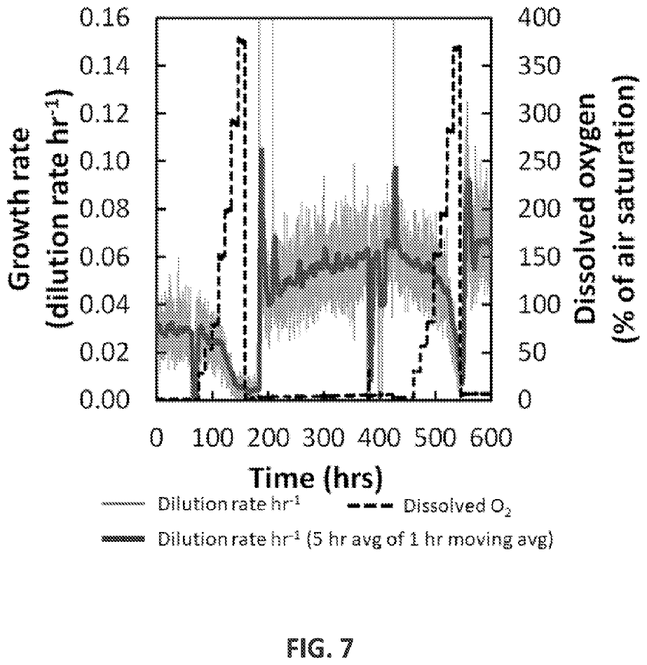

A wild-type Nitzschia Inconspicua strain GAI-229 was grown in a turbidostat under high light, low concentration conditions where there was little self-shading to reduce the light intensity. The light intensity was 230 microEinsteins per meter squared per second. The growth rate per hour was measured based on the dilution rate to maintain a constant algae concentration in the turbidostat. The oxygen was gradually increased until the algae stopped growing, then decreased so that the algae began growing again as illustrated in FIG. 7. Eventually growth was completely inhibited by the O₂ concentration and the high-O₂ selection pressure was removed to allow the culture to recover. Upon repeating this procedure, the culture grew more rapidly during the entire high-O₂ selection procedure. The high-O₂ pressure was repeated again after a recovery period and no additional improvement was observed in high-O₂ tolerance or growth so the resulting culture was collected and designated as the high-O₂ tolerant Nitzschia inconspicua strain GAI-337.

FIG. 1 presents the dependence of growth rate on dissolved oxygen concentration for the wild-type strain. FIG. 2 presents the dependence of growth rate on dissolved oxygen concentration for the new high-oxygen tolerant strain. FIG. 3 presents the growth rate of the new high-oxygen tolerant strain and the wild-type strain grown at a pH of approximately 9.3 in 0.3 molar sodium as carbonate/bicarbonate. The new strain exhibits much better oxygen tolerance (see FIG. 1 and FIG. 2), and the new strain exhibits much higher productivity under high or low oxygen conditions at a pH of 9.3 (see FIG. 3). The higher productivity was maintained at pH 10 as illustrated in FIG. 4.

This unexpected combination of growing a high oxygen tolerant strain at pH of higher than 9 or higher than 10 to obtain much higher overall productivity than either method alone can be exploited to obtain high productivity in commercial cultivation systems. The essential components are to find or develop high oxygen tolerant strains, and then to cultivate these strains in a bicarbonate/carbonate media at high pH. The high pH defined as at least 9 when carbon dioxide is added to the system or at least 10 for direct air capture of carbon dioxide.

### Example 3

In this study, a high O₂ tolerant strain of Nitzschia inconspicua was produced by growing a dilute turbidostat culture in increasingly high oxygen concentrations until growth was completely inhibited. After growth inhibition, the cultures were returned to a low O₂ state to recover growth, and put through repeated rounds of high O₂ stress. Three rounds of high O₂ stress yielded a strain with superior growth characteristics in both the presence and absence of high 02.

### Methods

### Strain Isolation and Identification

Nitzschia inconspicua str. hildebrandi GAI-229 was collected by Global Algae Innovations from the tidal area of a stream on the island of Kauai, Hawaii, USA. The strain was identified as a diatom from the genus Nitzschia by physiological and genetic characteristics (see Oliver et al. 2021).

### Generation of High-Oxygen Strain GAI-337

The wild-type strain Nitzschia inconspicua str. hildebrandi GAI-229 was placed into a turbidostat photobioreactor under constant temperature (24° C.), pH>9 and light at a greater intensity than the saturating intensity for the strain. The O₂ concentration in the sparging gas was gradually increased, leading to high O₂ selective pressure while simultaneously selecting for culture growth (slower growing cells were quickly diluted out of the culture). As the O₂ concentration approached ^{~}80% of the sparging gas (^{~}380% air saturation as measured in the liquid), growth was completely inhibited. At that point, the O₂ concentration in the sparging gas was decreased and the culture was allowed to recover for 12.5 days. This procedure was repeated 3 consecutive times and the improvement in growth was monitored in relation to the O₂ concentration. The high O₂ adaptation appeared within the strain during the first round of this process, and additional rounds of attempted directed evolution afterward did not appear to lead to additional improvement.

### Strain Cultivation and Maintenance

### Culture Medium

Strains were grown in brackish media as described previously (Quigg and genome papers). All culture medium was filter sterilized using Nalgene Rapid-Flow bottle top vacuum filters (Nalgene, USA).

### Culture Growth and Maintenance Prior to Experimentation

20 mL cultures were initially grown in 50 mL flasks and scaled up to 400 mL cultures in 1 L flasks prior to inoculation in flat-sided bottles. Flask cultures were grown at room temperature (^{~}23° C.) on orbital shaker tables (110 RPM) with constant light (100 μmol photons m⁻² s⁻¹). Cultures were regularly diluted as necessary to maintain growth in the flasks without nutrient or light limitation.

After a suitable amount of biomass was accumulated, 400 mL cultures were transferred to pond-mimicking conditions in 500 mL flat-sided bottles (No. 1396, Pyrex, Germany) with stainless steel bubble tubes extending 2 cm below the culture surface. Inlet and outlet gas was filter-sterilized by Acro 37 TF vent devices (Pall Corporation, USA). Inlet gas was provided by aquarium pumps or by mass flow controllers. Cultures were illuminated by cool white LEDs with water-cooled heatsinks attached to the back of the LED panels. The diel light regime was programed to mimic a solar day in Kauai in mid-June (29.8 MJ m⁻² d⁻¹) with the light intensity measured by an integrating sphere in the middle of a water-filled experimental bottle. The maximum light intensity during the light cycle was 2,200 μmol photons m⁻² s⁻¹ as measured by the spherical probe, which corresponded to 2,300 μmol photons m⁻² s⁻¹ as measured by a flat probe placed against the glass inside the center of an empty experimental bottle. 50 mm computer fans were positioned to blow cooling air between the LEDs and the culture bottles. Culture temperatures were either allowed to fluctuate naturally, being heated by the lights and cooled by the ambient air, which mimicked natural temperature fluctuations seen in outdoor ponds in June in Kauai, or controlled by a programmable water bath connected to a plexiglass enclosure housing the culture bottles. After transfer to pond-mimicking flat-sided bottles, cultures were allowed to acclimate for three to seven days prior to the start of an experiment.

### Strain Storage and Revival

Strains were grown in shaker flasks as above, then frozen by the methods of Elliot et al. (2012) at −80° C. Briefly, 1.9 mL of culture was transferred to a 2 mL Nalgene cryogenic tube in a sterile hood. 100 μL of DMSO (brand) was added, the tube was closed and gently inverted several times and quickly placed into a pre-chilled (4° C.) Mr. Frosty (Nalgene, USA) which was then quickly placed into a −80° C. freezer. The tubes were kept at −80° C. until revival. For revival, the tube was held in a water bath at 37° C., inverted occasionally until no ice remained, then centrifuged for 1 minute at 1000 RPM. The supernatant was quickly aspirated and the cells were resuspended in fresh culture medium. After a second round of centrifugation and aspiration of the supernatant as above, the cells were resuspended in fresh culture medium and transferred to a 50 mL shaker flask with a total medium volume of 15 mL. The flask was kept in low light on a benchtop for 1 day, then covered with a Kimwipe® to provide shade, and placed on an orbital shaker table. After the culture showed signs of growth (1-2 weeks depending on the strain and frozen culture density), the shade was removed. After several days of growth, the entire culture was transferred to a 250 mL flask and the volume was set to 100 mL with fresh nutrient enriched culture medium.

### Experimental Diel-Cycle Pond-Mimicking Culture Conditions

Triplicate 400 mL cultures were grown in 500 mL flat-sided bottles as described above with temperature control provided by a plexiglass water bath connected to a programmable water bath set to mimic the diel temperature swings seen in outdoor pond cultures in Kauai. For different maximum temperature experiments, the temperature curve was adjusted higher or lower with the minimum temperature (24° C.) remaining the same. Cultures were bubbled with house air regulated by a mass flow controller set to deliver the required air-flow rates as indicated. Cultures were sampled in situ through a side port comprised of a tube clamp and one-way check valve connected to the bubble tube. To sample, the gas line above the sample port was clamped, culture was drawn through the bubble tube and sample port to flush the line with culture and discarded, then the sample was extracted. Due to the rapid settling rate of this diatom strain, in situ sampling was employed as the best method for consistent sampling. Cultures were sampled and diluted with fresh medium once per day just after sunset (dusk). Cultures were diluted to 1.25 OD₇₅₀ (^{~}0.45 g L⁻¹ AFDW) unless otherwise indicated.

### High Oxygen Stress Diel-Cycle Conditions

Cultures grown to maximize oxygen stress were cultured as above, but without bubbling during the day to maximize the amount of photosynthetically-produced oxygen in the bottles. The cultures were bubbled at night with air (100 mL min⁻¹ bottle⁻¹) to prevent anaerobic culture conditions. The OD₇₅₀ after dilution for these experiments was set to 0.8. Temperatures were allowed to vary naturally as described above with a low temperature at night around 23° C. and a maximum temperature during the day around 33° C.

### OD₇₅₀

One mL of culture was rigorously pipetted to break apart any clumps within the sample, then 100 mL was mixed with 900 mL fresh culture medium in a cuvette and again pipetted rigorously. The optical density at 750 nm (OD₇₅₀) was measured using a DU800 UV-visible spectrophotometer (Beckman Coulter, USA) blanked with fresh culture medium. The OD₇₅₀ was read three times for each sample, pipetting the sample quickly just prior to each measurement, and the mean of the three measurements was recorded. This method was necessary to improve the accuracy of the OD₇₅₀ measurements because the cells settled rapidly and exhibited variability in light scattering for each scan.

### Ash-Free Dry Weight (AFDW)

10 mL of culture was transferred to a 15 mL falcon tube and centrifuged at 3000 RPM for 5 minutes at room temperature. The supernatant was discarded, and the pellet resuspended in 1 mL of 0.5 M ammonium formate by gently pipetting 6-8 times. 10 additional mL of ammonium formate was added and the 1 mL pipette was rinsed in the cell suspension to remove as many of the cells as possible. The tube was centrifuged again as above and the supernatant was discarded. The cell pellet was again suspended in 1 mL of 0.5 M ammonium formate and vacuum filtered onto a pre-ashed (550° C. for 30 minutes) GF/F glass fiber filter (Whatman, United Kingdom) loaded into a vacuum filtration unit connected to a vacuum pump. The remaining cells were rinsed from the falcon tube with 1 mL 0.5 M ammonium formate and applied to the filter. The filters were placed into aluminum trays and dried at 105° C. overnight. The dry filters were weighed to yield dry weights, then ashed at 550° C. for 2 hours. The ash free filters were then placed into a 105° C. oven overnight, and quickly removed and weighed. AFDW was calculated as the difference between the dry weight and the filter weight after ashing.

### Pigment Analysis

Chlorophylls a and c1+c2 were quantified using the spectrophotometric method of Ritchie (2006). 100 μL of culture was centrifuged at 8,000 g for 5 minutes at 4° C., the supernatant was aspirated, and the pellets were frozen at −20° C. Samples were placed on ice in the dark and extracted with 1 mL of 100% methanol for 20 minutes followed by centrifugation at 15,000 rpm for 10 minutes at 4° C. 0.7 mL of the extract was transferred to a cuvette and the absorbance was measured at 632 and 665 nm.

### Fatty Acid Methyl Ester (FAME) Analysis

Lipids were quantified by GC-FID FAME analysis using the methods of Work et a. (2010). 0.5 mL of culture was centrifuged at 8,000 g for 5 minutes at 4° C., the supernatant was aspirated, and the pellet was frozen at −20° C. The pellet was thawed, and the lipids were saponified by resuspension in 1 mL methanol saturated with NaOH and heated in sealed vials at 100° C. for 90 minutes. The vials were cooled to room temperature and acid-catalyzed methylation was achieved by adding 1.5 mL of methylation reagent (14.6 mL 12 N HCl+235.4 mL MeOH) and incubated at 60° C. overnight. FAMEs were extracted by adding 1.25 mL n-Hexane and gently inverting for 30 minutes on a shaker table. Extracts were analyzed by gas chromatography-flame ionization detection (GC-FID) using an Agilent 7890A gas chromatograph with a DB5-ms column (Agilent Technologies, Santa Clara, CA) and quantified by comparison with a Supelco 37 component FAME standard (Sigma-Aldrich, USA).

### Protein Extraction and Quantification by Modified Lowry

Cultures were sampled in-situ and 0.5 mL aliquots were centrifuged at 5,000 g for 5 minutes, the supernatants were aspirated, and the pellets were frozen at −80° C. for later analysis. Pellets were resuspended in lysis buffer comprised of 50 mM Tris-HCl pH 8, 75 mM NaCl, 5% glycerol, and 1% SDS. Resuspended samples were kept on ice and probe sonicated for 30 seconds in ice water and returned to ice between rounds of sonication. Three rounds of sonication were performed per sample. Sonicated samples were centrifuged at 15,000 RPM, 4° C. for 10 minutes. The supernatant was then analyzed using the Bio-Rad DC modified Lowry protein assay kit using the manufacturer's specifications.

### Carbohydrate Analysis

0.5 mL of culture was removed from the culture bottle and immediately frozen at −20° C. For each sample, 900 μl of anthrone reagent (2 g L⁻¹ anthrone, 71% 10 N H₂SO₄+29% H₂O v/v) was aliquoted into a 1.7 mL tube and chilled in an ice water bath. 100 μl of thawed sample was added to the chilled anthrone aliquots and mixed by inversion. Once all samples and D-glucose standards (0-500 mg d-glucose L⁻¹ in H₂O) were prepared, the tubes were transferred simultaneously to a boiling water bath and left for exactly 12 minutes. The tubes were then simultaneously returned to the ice water bath to stop the reaction. The tubes were vortexed, 200 μL of each sample was transferred to a 96-well plate, and the absorbance at 625 nm was measured in a Synergy 2 plate reader (Bio Tek, USA).

### Calculation of Gallons of Gasoline Equivalent (GGE)

Based on DOE guidelines, FAMEs ton⁻¹ AFDW were multiplied by 0.280 and carbohydrates ton⁻¹ AFDW were multiplied by 0.106 and summed to yield GGE ton⁻¹ AFDW. Because protein makes up a significant proportion of algal biomass, the inclusion of the industrially feasible conversion of protein to fuel, using hydrothermal liquefaction (Valdez et al. 2014) or deamination and conversion to C4 and C5 alcohols (Huo et al. 2011), was also added to the analysis with protein ton⁻¹ AFDW multiplied by 0.1.

### Results

Generation of the high-oxygen tolerant strain GAI-337 from the wild-type strain GAI-229

Selection pressure for high O₂ tolerance in Nitzschia inconspicua str. hildebrandi GAI-229 was achieved by growing the strain in a dilute turbidostat culture and gradually increasing the O₂ concentration in the sparging gas, which led to decreased growth rates (dilution rates) (see FIG. 7). Eventually growth was completely inhibited by the O₂ concentration and the high-O₂ selection pressure was removed to allow the culture to recover (between hours 160 and 460, FIG. 7). Upon repeating this procedure, the culture grew more rapidly during the entire high-O₂ selection procedure (hours 460 to 545, FIG. 7). The high-O₂ pressure was repeated again after a recovery period, and no additional improvement was observed in high-O₂ tolerance or growth so the resulting culture was collected and designated as the high-O₂ tolerant strain GAI-337.

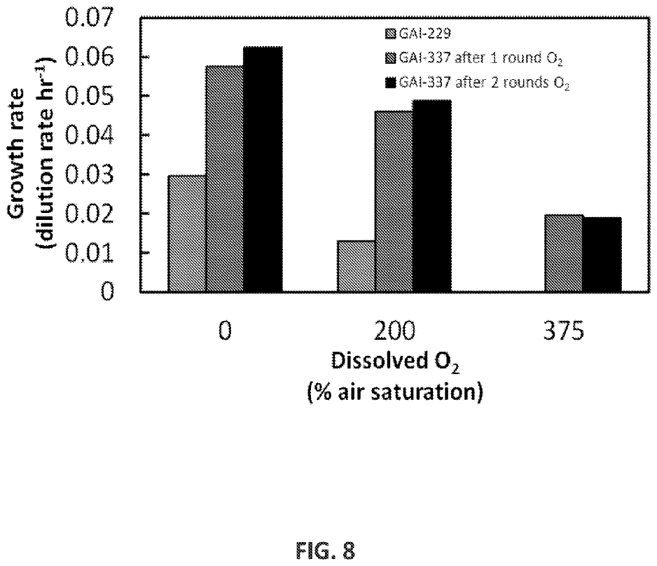

During the process of generating the high-O₂ tolerant strain GAI-337, growth rates of the wild-type strain GAI-229 and the high-O₂ tolerant strain were compared at different dissolved O₂ concentrations (FIG. 8). At elevated O₂ concentrations, GAI-337 showed improved growth rates over the parent strain (FIG. 8). Interestingly, when the cultures were grown under conditions where O₂ was being removed by sparging (zero O₂ bars on the left of FIG. 8), GAI-337 exhibited ^{~}two-fold higher growth rates than the wild-type GAI-229. These results suggest that the high-O₂ tolerant strain GAI-337 possessed improved O₂ tolerance as well as improved growth rates regardless of the presence of O₂.

Comparison of Wild-Type GAI-229 with the High-Oxygen Tolerant Strain GAI-337

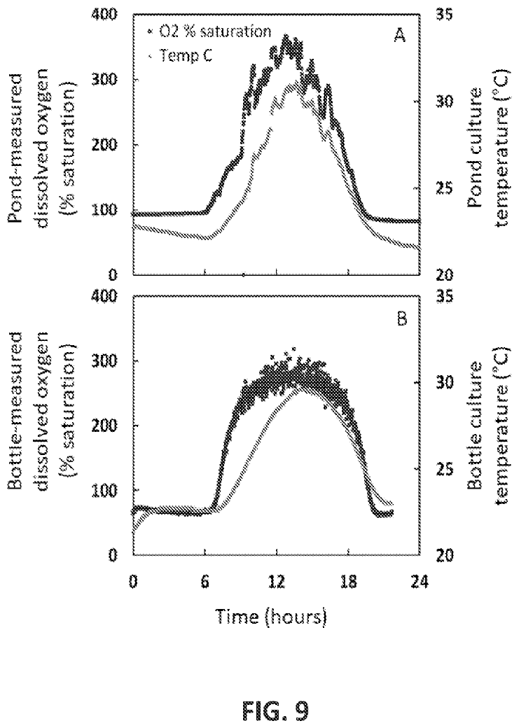

To compare the strains in conditions approximating outdoor industrial ponds, GAI-337 and GAI-229 were tested under high O₂ stress conditions with pond-mimicking diel light and temperature (see FIG. 9 for temperature and O₂ curves in outdoor ponds (A) compared with the pond-mimicking photobioreactors (B)). Areal productivities of AFDW were measured for the two strains grown in biological triplicate over four consecutive days, with daily dilutions to a set OD₇₅₀ of 0.8 just after sunset. High O₂ was obtained naturally by photosynthetic oxygen evolution of the cultures in the bottles without bubbling. Using a probe to measure the dissolved O₂ concentration, photosynthesis quickly (within 1 hour of sunrise) caused the dissolved O₂ concentration to saturate the medium beyond the probe's operating capabilities (over 300% saturation compared to air, see FIG. 9B). The cultures were bubbled with air during the night to prevent anaerobic conditions, keeping the dissolved O₂ concentration above ^{~}60%. Under these high O₂ stress pond-mimicking conditions, GAI-337 significantly outperformed the wild-type strain in areal biomass production (p<0.001) with an 89% increase in g AFDW m⁻² d⁻¹ (FIG. 4). This result confirmed enhanced biomass productivity in the mutant strain GAI-337 compared to the wild-type strain GAI-229 when placed into high O₂ pond-mimicking conditions, which are expected in photosynthetically active outdoor ponds.

Assessment of Initial Culture Density after Dilution on Areal Biomass Productivity

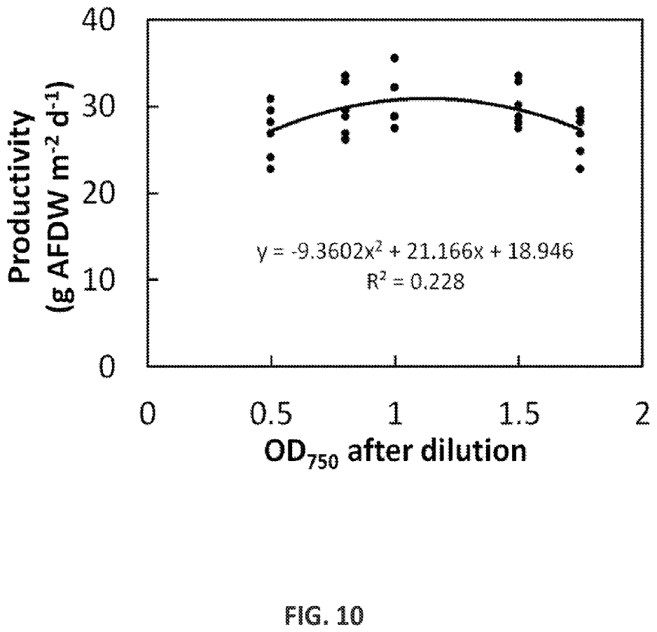

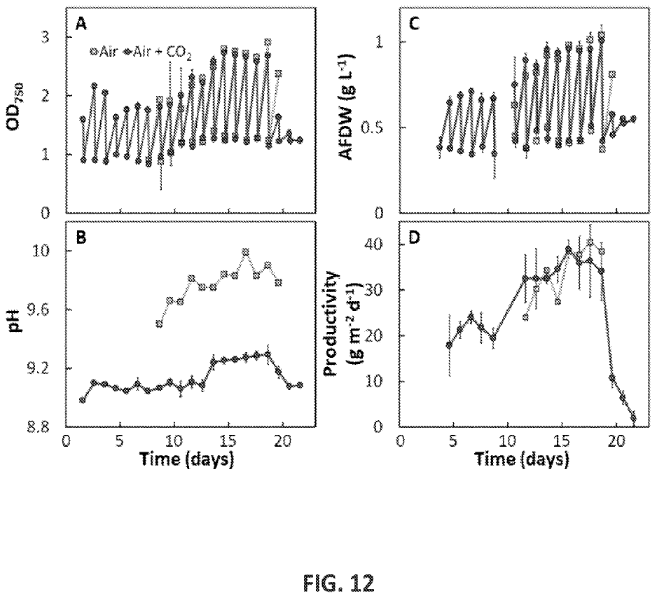

To assess the effect of the starting culture density on areal productivity of GAI-337, a series of experiments were performed where cultures were placed in pond-mimicking conditions, diluted just after sunset to different culture densities, and areal productivity was measured. Culture density setpoints were determined by OD₇₅₀. These experiments showed that the optimal culture density was between 1 and 1.5 OD₇₅₀ (FIG. 10), which corresponded to an initial culture density after dilution of about 0.45 g AFDW L⁻¹. This result was further supported in the experiment shown in FIG. 12 where the dilution setpoints were initially set to 0.8 OD₇₅₀ and then switched to 1.25 on day 12, leading to significantly higher productivities (p<0.001).

Assessment of Initial Culture Density after Dilution and pH Control on Areal Biomass Productivity and Nutrient Limitation to Induce Lipid Formation

To assess the productivity improvements made by optimizing the initial culture density and to test the effect of bubbling with and without supplemental CO₂, and hence, pH had on productivities, an experiment was performed where the cultures were initially set to a culture density setpoint of 0.8 OD₇₅₀ (0.37 g AFDW L⁻¹) and run for 12 days, then the setpoint was increased to 1.25 (0.45 g AFDW L⁻¹) (FIGS. 12A and 12C). Three cultures were grown with 0.5% CO₂ supplementation in air to keep the pH between ^{~}9.1 and 9.3 and one culture was grown with air bubbling alone which allowed pH to fluctuate (FIG. 12B). pH was measured at dusk before dilution with fresh medium so the pH values shown in FIG. 12B show the endpoint of the pH after each day's growth and does not depict the pH changes caused by dilution with fresh medium. Finally, for the last three days of the experiment, the culture medium used for dilution was changed to a nutrient limited formulation to assess lipid formation by GAI-337 under pond-mimicking conditions. The increase in the daily OD₇₅₀ setpoint after dilution significantly increased areal productivity (FIG. 12D), leading to an average productivity of ^{~}34 g AFDW m⁻² d-1, which places GAI-337 among the most productive diatoms reported in the literature under pond-mimicking conditions. Interestingly, due to the bicarbonate-rich medium formulation, supplemental CO₂ did not affect productivities (FIG. 5). The pH of the cultures grown with CO₂ supplementation were between 9.1 and 9.3, whereas the culture without CO₂ was around 9.8 during maximal productivity after the switch to 1.25 initial OD₇₅₀.

Table 1 shows the concentration and areal productivities of ash free biomass, protein, carbohydrates, and FAMEs for cultures growing under the high productivity conditions shown in FIG. 12, with samples taken for proximate analysis on day 18.

TABLE 1
Mean concentration and areal productivity ±1 standard deviation
of ash free biomass, protein, carbohydrates, and FAMEs for
GAI-337 grown under pond-mimicking conditions in biological
triplicate cultures continuously bubbled with air supplemented
with 0.5% CO2 at a rate of 100 mL min − 1 per bottle.

| | | Areal |
|---|---|---|
| | Concentration | productivity |
| Component | (g L⁻¹) | (g m⁻² d⁻¹) |
| AFDW | 0.96 ± 0.1 | 36.4 ± 7.9 |
| Protein | 0.53 ± 0.03 | 20.1 ± 1.2 |
| Carbohydrate | 0.34 ± 0.03 | 12.9 ± 1.0 |
| FAME | 0.14 ± 0.02 | 5.3 ± 0.8 |

### Assessment of the Effect of O₂ Concentration and Temperature on Areal Biomass Productivity Under Pond-Mimicking Conditions

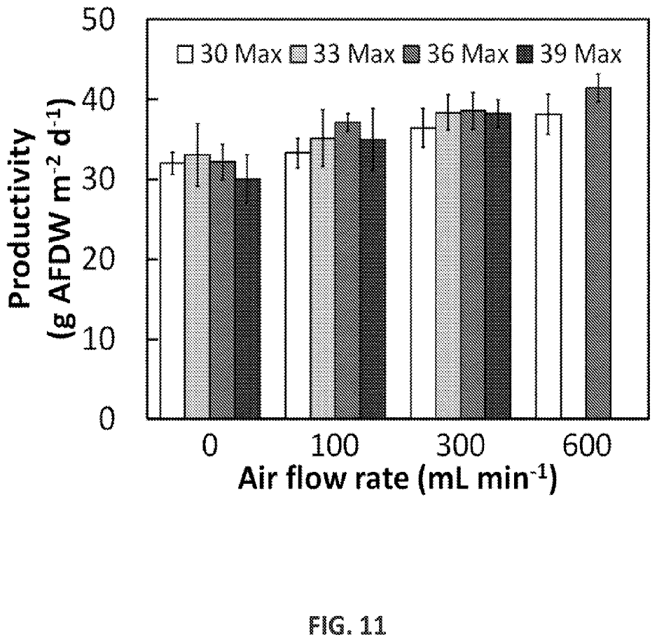

To assess the effect O₂ concentration and temperature had on areal productivity, a series of experiments were done under diel light and temperature regimes mimicking outdoor ponds where the air flow rate and maximum temperature during the day were varied. Triplicate cultures were tested over three days for each condition. Input air was first bubbled through a hydrator prior to injection into the cultures, which eliminated evaporative losses as determined by initial and final bottle+culture masses (data not shown). Cultures grown without air bubbling were bubbled with 100 mL min⁻¹ bottle⁻¹ air at night to prevent anaerobic culture conditions, while air flow at the rate indicated was maintained day and night for the other conditions. As air flow was increased, higher areal productivities were achieved (FIG. 11). As demonstrated in FIG. 5, CO₂ supplementation did not affect productivity, therefore the increase in productivity with higher air flow rates was likely a consequence of decreased O₂ concentrations due to removal by bubbling, rather than increased CO₂ availability due to the higher air flow rate. Higher temperatures increased areal productivities up to 36° C., beyond which (39° C.) high temperatures appeared to hinder growth (FIG. 11). These experiments demonstrate that while the new strain GAI-337 was more tolerant to high O₂ than the parent strain GAI-229 (FIGS. 8 and 4), O₂ concentration in the culture medium still played an inhibitory role on productivity under pond-mimicking conditions. These data suggest that GAI-337 would be most suited to a relatively warm climate, similar to the southwestern Texas desert conditions studied by Chagoya et al. (2014) (23-37° C. diel temperatures), and would benefit from pond management strategies that remove some of the O₂ in the cultures, though productivities in this strain were excellent even under the harshest O₂ conditions tested. When put into optimal pond-mimicking conditions, GAI-337 achieved maximal productivities of 41.5±1.7 (n=9) g AFDW m⁻² d⁻¹, a highly competitive achievement for a diatom and an encouraging result for the economic feasibility of this strain.

### Discussion

Industrial outdoor microalgal ponds experience high O₂ concentrations due to the rapid release of photosynthetically produced O₂ by the algae. These concentrations quickly rise to the point where areal productivity is reduced due to the competitive inhibition of RuBisCO and increased deleterious consequences from reactive oxygen species, leading to decreased light use efficiency during peak insolation during the day. While pond management strategies to outgas O₂ are feasible, improvement of O₂ tolerance of industrial strains is also an attractive, and little explored option. In this study, a high O₂ tolerant strain of Nitzschia inconspicua strain hildebrandi was generated by selection under three rounds of gradually increasing O₂ conditions followed by recovery periods. The resulting strain, GAI-337, was compared to the wild-type strain (GAI-229) over a range of dissolved O₂ concentrations in dilute turbidostat cultures and then under high-O₂ stress pond-mimicking conditions. The mutant GAI-337 strain achieved 89% higher aerial productivity as measured in g AFDW m-2 d-1 relative to the wild-type strain. Interestingly, GAI-337 exhibited faster growth rates than the wild-type strain, even at low O₂ concentrations, suggesting that the adaptation achieved by GAI-337 improved not only the O₂ tolerance of the strain, but the growth rate as well.

In order to investigate the areal productivity potential of GAI-337, a series of experiments focused on dilution and harvesting time, initial culture density, CO₂ infusion and therefore pH, O₂ concentration, and temperature were conducted. It was found that harvesting just after sunset and cultures set to an initial OD₇₅₀ of 1.25 (^{~}0.45 g L⁻¹ AFDW) after dilution yielded the highest areal productivities. Due to the high bicarbonate nature of the culture medium tested, CO₂ infusion had no beneficial effect on areal biomass productivities and the resulting pH differences also had no effect. This is encouraging because while colocalization of algal production facilities with CO₂ point sources such as power plants could directly divert CO₂ release into the atmosphere, this process is costly and point sources co-located with suitable environments for algal production are limited. The lack of necessity for pH management by bubbling with CO₂ for maximal productivity, also simplifies pond management and design. While the O₂ tolerance of GAI-337 was improved over the wild-type strain, O₂ removal still improved areal productivities, and coupled with an optimal pond-mimicking temperature regime, led to 41.5 g AFDW m-2 d-1 areal productivity. This result in lab-based pond-like conditions is close to those seen for the elite biomass producer Picochlorum celeri by Krishnan et al. (2021) (45.2 g AFDW m-2 d-1) and places GAI-337 among the most productive diatoms known, even if a similar decrease between lab vs outdoor pond productivities is realized as was seen for P. celeri. While species of Picochlorum grow rapidly under replete medium conditions, under nutrient stress, they accumulate carbohydrates (Dahlin et al. 2019) whereas GAI-337 accumulates lipids, adding to the diversity of energy storage molecules attainable in industrially-relevant high-productivity algal strains.

In this study, nutrient limitation shifted the biomass composition of GAI-337 to a GGE ton-1 AFDW of 157, at the expense of areal productivity. This result suggests that a biphasic production process would yield the highest overall light use efficiency for fuel production, where rapidly growing nutrient replete cultures yield high biomass during the first phase, which is then fed into nutrient limited cultures for “polishing” the biomass composition during the second phase.

### REFERENCES CITED

1. Marquez, Faacundo, Ken Sasaki, Naomichi Nishioi, and Shiro Nagal. Inhibitory effect of oxygen accumulation on the growth of Spirulina Platensis. Biotechnology Letters, Vol. 17 No. 2, p 2250228. 1995.
2. Weissman, Joseph, Raymond Goebel, and John Benemann. Photobioreactor Design: Mixing, Carbon Utilization, and Oxygen Accumulation. Biotechnology and Bioengineering, Vol 31. P 336-344. 1988.
3. Wagner, Fred; and B. Welch. Oxygen-tolerant Strain of Chlorella sorokiniana. Applied Microbiology p 139-144. January 1969.
4. Gao, Song; Scott Edmundson; and Michael Huesemann. Oxygen stress mitigation for microalgal biomass productivity improvement in outdoor raceway ponds. Algal Research Vol 68, p1-8. 2022.
5. Oliver, Aaron, Sheila Podell, Pinowska Agnieszka, Jesse C Traller, Sarah R Smith, Ryan McClure, BeliaevAlex, et al. 2021. “Diploid Genomic Architecture of Nitzschia Inconspicua, an Elite Biomass Production Diatom.” Scientific Reports (Nature Publisher Group) 11 (1). https://doi.org/10.1038/s41598-021-95106-3.
6. Chagoya, Jennifer C, Jola Brown, Michael S Gomez, Junling Zhang, Yuelu Jiang, Katherine Laverty, Lou Brown, Antonietta Quigg, and Mark D Burow. 2014. “Media Optimization and Lipid Formation of Two Native Diatoms for Cultivation in the Southwest Texas Desert.” Journal of Applied Phycology 26 (5): 2075-85. https://doi.org/10.1007/s10811-014-0238-1.
7. Elliott, Lee G., Corinne Feehan, Lieve M. L. Laurens, Philip T. Pienkos, Al Darzins, and Matthew C. Posewitz. 2012. “Establishment of a Bioenergy-Focused Microalgal Culture Collection.” Algal Research 1 (2): 102-13. https://doi.org/10.1016/j.algal.2012.05.002.
8. Work, Victoria H., Randor Radakovits, Robert E. Jinkerson, Jonathan E. Meuser, Lee G. Elliott, David J. Vinyard, Lieve M. L. Laurens, G. Charles Dismukes, and Matthew C. Posewitz. 2010. “Increased Lipid Accumulation in the Chlamydomonas Reinhardtii sta7-10 Starchless Isoamylase Mutant and Increased Carbohydrate Synthesis in Complemented Strains.” Eukaryotic Cell, August. https://doi.org/10.1128/EC.00075-10.
9. Valdez, Peter J., Vincent J. Tocco, and Phillip E. Savage. 2014. “A General Kinetic Model for the Hydrothermal Liquefaction of Microalgae.” Bioresource Technology 163 (July): 123-27. https://doi.org/10.1016/j.biortech.2014.04.013.
10. Hildebrand, Mark, Aubrey K Davis, Sarah R Smith, Jesse C Traller, and Raffaela Abbriano. 2012. “The Place of Diatoms in the Biofuels Industry.” Biofuels 3 (2): 221-40. https://doi.org/10.4155/bfs.11.157.
11. Krishnan, Anagha, Maria Likhogrud, Melissa Cano, Scott Edmundson, Jenna B Melanson, Michael Huesemann, John McGowen, Joseph C Weissman, and Matthew C Posewitz. 2021. “Picochlorum Celeri as a Model System for Robust Outdoor Algal Growth in Seawater.” Scientific Reports 11 (1): 11649-11649. https://doi.org/10.1038/s41598-021-91106-5.
12. Dahlin, Lukas R., Alida T. Gerritsen, Calvin A. Henard, Stefanie Van Wychen, Jeffrey G. Linger, Yuliya Kunde, Blake T. Hovde, Shawn R. Starkenburg, Matthew C. Posewitz, and Michael T. Guarnieri. 2019. “Development of a High-Productivity, Halophilic, Thermotolerant Microalga Picochlorum Renovo.” Communications Biology 2 (1): 1-9. https://doi.org/10.1038/s42003-019-0620-2.

We claim:

1. A method of photosynthetically or mixotrophically cultivating a high oxygen tolerant algae strain, the method comprising cultivating the strain in an aqueous medium comprising a pH of greater than 9.

2. The method of claim 1 there the aqueous medium pH during cultivation is greater than 10.

3. The method of claim 1, wherein the strain has less than an about 40% reduction of growth rate at about 24 mg/L of dissolved oxygen concentration relative to the growth rate at about 8 mg/L dissolved oxygen concentration.

4. The method of claim 1, wherein the strain has less than an about 20% reduction of growth rate at about 16 mg/L of dissolved oxygen concentration relative to the growth rate at about 8 mg/L dissolved oxygen concentration.

5. The method of claim 1, wherein the high oxygen tolerant algae strain has less than an about 40% reduction of growth rate at about 24 mg/L of dissolved oxygen concentration relative to the growth rate at about 8 mg/L dissolved oxygen concentration.

6. The method of claim 1, wherein the aqueous medium comprises at least 0.1 molar concentration of carbonate and bicarbonate ions combined.

7. The method of claim 6, wherein the carbonate comprises at least one of sodium carbonate and potassium carbonate, and wherein the bicarbonate comprises at least one of sodium bicarbonate and potassium bicarbonate.

8. The method of claim 1, wherein the high oxygen tolerant algae strain is cultivated in a photobioreactor.

9. A Nitzchia inconspicua strain GAI-337.

10. A method of producing a high oxygen tolerant algae strain, the method comprising:
a) growing a cell culture of a parent algae strain in a medium comprising a pH of at least 9.0;
b) gradually increasing oxygen concentration in the medium while diluting slow-growing cells out of the medium until cell growth is inhibited;
c) decreasing the oxygen concentration in the medium;
d) allowing the cell growth to recover;
e) repeating steps a) through d) at least one time;
wherein after step e), the cell culture comprises the oxygen tolerant algae strain.

11. The method of claim 10, wherein steps a) through d) are repeated one time.

12. The method of claim 10, wherein the light intensity during the cell culture growth is greater than the saturating intensity of the parent algae strain.

13. (canceled)

14. The method of claim 10, wherein the pH of the medium is between 9 and 11.

15. The method of claim 10, wherein the method is done in a photobioreactor.

16. (canceled)

17. The method of claim 10, wherein the high oxygen tolerant algae strain has less than an about 40% reduction of growth rate at about 24 mg/L of dissolved oxygen concentration relative to the growth rate at about 8 mg/L dissolved oxygen concentration.

18. The method of claim 10, wherein the high oxygen tolerant algae strain has less than an about 20% reduction of growth rate at about 16 mg/L of dissolved oxygen concentration relative to the growth rate at about 8 mg/L dissolved oxygen concentration.

19. The method of claim 10, wherein the parent algae strain is a wild-type strain.

20. The method of claim 10, wherein the parent algae strain is a Nitzschia strain.

21. The method of claim 20, wherein the parent algae strain is Nitzschia inconspicua.

22. A high oxygen tolerant algae strain produced by the method of any one of claim 10.
